(19) United States
(12) Patent Application Publication
(10) Pub. No.: US 2007/0298101 A1
(43) Pub. Date: Dec. 27, 2007

(54) Controlled-Release Formulation Comprising Tamsulosin Hydrochloride

(75) Inventors: Lech Wiackowski, Warszawa (PL); Beata Wiackowska, Warszawa (PL); Wieslaw Szelejewski, Warszawa (PL); Andrzej Zaremba, Warszawa (PL); Edyta Pesta-Dynda, Warszawa (PL); Zofia Marchlewska-Cela, Piastow (PL)

Correspondence Address: MATTHIAS SCHOLL, 14781 MEMORIAL DRIVE, SUITE 1319, HOUSTON, TX, 77079, US

(73) Assignee: INSTYTUT FARMACEUTYCZNY, WARSZAWA (PL)

(21) Appl. No.: 11/573,562

(22) PCT Filed: Aug. 12, 2005
(86) PCT No.: PCT/PL05/00052

§ 371 Date: Aug. 2, 2007

(30) Foreign Application Priority Data
Aug. 12, 2004 (PL) P-369566

(51) Int. Cl. A61K 9/28; A61K 31/18

(52) U.S. Cl. 424/465; 424/474; 514/603

(57) ABSTRACT

Taught herein is a solid, oral, controlled-release pharmaceutical composition of tamsulosin hydrochloride in the form of an enteric-coated tablet, wherein tamsulosin hydrochloride is homogenously dispersed within a matrix consisting of a mixture of a fatty component and a hydrophilic component, together with at least one diluent, and optionally other pharmaceutically acceptable excipients, exhibiting the following dissolution profile of tamsulosin hydrochloride, as measured in a Type II paddle apparatus in accordance with the dissolution testing method specified in the European Pharmacopoeia, i.e., at 37±0.5° C. and 100 rpm in a 0.1 N HCl buffer for 2 hours, followed by pH 7.2 buffer for the rest of the test: 10-40% dissolution during first 2 hours (in HCl), 35-70% dissolution after 3 h (in pH 7.2 buffer system), not less than 70% dissolution of the declared content after 5 h (in pH 7.2 buffer system).

### CROSS-REFERENCE TO RELATED APPLICATIONS

This is a National Stage Application of International Patent Application No. PCT/PL 2005/000052, with an international filing date of Aug. 12, 2005, which is based on a Polish Patent Application No. P-369566, filed Aug. 12, 2004. The contents of both of these specifications are incorporated herein by reference.

### BACKGROUND OF THE INVENTION

1. Field of the Invention

This invention relates to a solid oral controlled-release pharmaceutical composition comprising tamsulosin hydrochloride.

2. Description of the Prior Art

Tamsulosin HCl, (−)-(R)-5-[2-[[2-(o-ethoxyphenoxy)ethyl]amino]propyl]-2-methoxybenzenesulfonamide monohydrochloride, is a selective, competitive antagonist of type α1A post-synaptic adrenergic receptors in the prostate, used to treat the symptoms of benign prostatic hyperplasia.

Due to its pharmacokinetic properties, tamsulosin hydrochloride is administered to patients in the form of an oral, controlled-release pharmaceutical composition, suitable for a once-daily administration.

The capsule formulation used in medical practice under the brand names Flomax®, Omnic® and Harnal® comprises pellets of tamsulosin hydrochloride (0.4 mg/capsule) as the main active ingredient and methacrylic acid co-polymer and microcrystalline cellulose as the main inactive ingredients.

European Patent Applications EP 0533297 A1 and EP 0194838 A1 disclose a pharmaceutical composition from which tamsulosin is gradually released due to the use of a matrix of a macromolecular, water-insoluble release-controlling agent selected from acrylic acid polymers and acrylic acid copolymers. This release-controlling agent is used as an aqueous suspension or emulsion or as a solution in an aqueous-organic system and acts also as a binder in the granulation step. Microcrystalline cellulose, used as a carrier, constitutes at least 50% by weight based on the weight of the pharmaceutical composition, and provides, on granulation, the cohesive particles. The particles containing tamsulosin hydrochloride are formed into tablets, capsules or granules. It is believed that the composition disclosed in EP 0533297 A1 covers the commercially available capsules Flomax®.

International Patent Publications WO 2004/043448, WO 2004/043449, WO 2004/056354 and WO 03/039530 disclose other pharmaceutical compositions of tamsulosin hydrochloride in the form of pellets and tablets, all of which use the acrylic polymers as release-controlling agents.

Although they are frequently used in pharmaceutical preparations as release controlling and film coating agents (Handbook of Pharmaceutical Excipients, 2nd Ed., 1994, p. 362-), methacrylic acid polymers and co-polymers are recently raising fears concerning the safety of mucous membranes of the gastrointestinal system. Therefore, their gradual withdrawal or at least, limiting exposure of patients to these compounds seems to be well-grounded.

In the International Patent Application Publication WO 94/06414 a hydrogel-type sustained-release pharmaceutical composition of tamsulosin hydrochloride which releases the active ingredient not only in the upper gastrointestinal tract, but also in the lower gastrointestinal tract, in particular in the colon, has been proposed. The pharmaceutical composition comprises a hydrophilic additive allowing for penetration of water into the core of the pharmaceutical composition as well as a hydrogel-forming polymer. An appropriate extent of gelling is achieved by using a hydrophilic substance that is dissolved in a small amount of water not to exceed 5 ml per 1 g of the active ingredient, such as polyvinylpyrrolidone, D-sorbitol or PEG 6000, in combination with a hydrogel-forming polymer characterized by its average molecular weight of over 2,000,000 or by viscosity of a 1% solution of not less than 1,000 cPs.

However, neither a hydrogel-type pharmaceutical composition nor any other tablet-form composition of tamsulosin hydrochloride has been brought into medical practice as of yet. Therefore, there is still a need for drug pharmaceutical compositions being an alternative to pellets that would be simple in manufacturing while having an equivalent in vitro release profile of the active ingredient and pharmacological activity.

### BRIEF DESCRIPTION OF THE DRAWINGS

The objects and advantages of the present invention will become more readily apparent after reading the ensuing description of the non-limiting illustrative embodiment and viewing the accompanying drawings, in which

FIG. 1 shows a dissolution profile of a pharmaceutical composition of tamsulosin hydrochloride in a tablet form according to one embodiment of the invention as compared to Omnic® capsules, for reference; and

FIG. 2 shows a dissolution profile of a pharmaceutical composition of tamsulosin hydrochloride in a tablet form according to another embodiment of the invention as compared to Omnic® capsules, for reference.

### DETAILED DESCRIPTION OF THE INVENTION

The objectives of the invention have been achieved by developing a solid oral controlled-release pharmaceutical composition in the form of enteric-coated tablets, exhibiting the following dissolution profile of tamsulosin hydrochloride, as measured in a Type II paddle apparatus in accordance with the dissolution testing method specified in the European Pharmacopoeia, i.e., at 37±0.5° C. and 100 rpm in a 0.1 N HCl buffer for 2 hours, followed by pH 7.2 buffer for the rest of the test:

10-40% dissolution during first 2 hours (in HCl),

35-70% dissolution after 3 h (in pH 7.2 buffer system), and

not less than 70% dissolution of the declared content after 5 h (in pH 7.2 buffer system).

The present invention relates to an enteric coated tablet pharmaceutical composition for a controlled release of tamsulosin hydrochloride wherein an active substance is homogenously dispersed within a matrix consisting of a mixture of a fatty component and a hydrophilic component, together with at least one diluent and optionally other pharmaceutically acceptable excipients.

The fatty component of the matrix is a natural or synthetic substance selected from the group consisting of C₁₂-C₁₈ long-chain fatty acids, glycerides of C₈-C₁₈ medium- and long-chain fatty acids; hydrogenated fatty oils such as castor oil; hydrogenated lecithins, and the mixtures thereof.

In a preferred embodiment of the invention, the fatty component of the matrix is a glyceride of a fatty acid, such as glycerol palmitostearate, glycerol monostearate, or glycerol behenate. The most preferred fatty component of the matrix is glycerol behenate.

Glycerol behenate, such as the commercially available Compritol 888 ATO, is a natural product being a mixture of 12-18% monoglycerides, 52-54% diglycerides and 28-32% triglycerides of docosanoic acid (over 87% of the fatty acid fractions). Glycerol behenate and other glycerides of fatty acids are used as tablet and capsule diluents and, in lower concentration, as lubricants. They may also form lipophilic matrixes for sustained-release tablet and microsphere pharmaceutical compositions.

The content of the fatty component of the matrix in the pharmaceutical composition according to the invention is 20-40% by weight based on the weight of the tablet's core.

The hydrophilic component of the matrix is any pharmaceutically-acceptable inert substance that would allow for penetration of water into the tablet's core, thereby swelling, gelling or thickening and forming a viscous layer facilitating the diffusion of an active substance. Suitable components of this type comprise, e.g., polyvinylpyrrolidone, polyvinyl alcohols, polyethylene glycols, ethers and esters of cellulose, preferably hydroxypropylmethylcellulose, sodium carboxymethylcellulose, alginates, and the mixtures thereof.

In a preferred embodiment of the invention, the hydrophilic component of the matrix is polyvinylpyrrolidone, in particular polyvinylpyrrolidone of a molecular weight within the range of 25,000 to 300,000. Particularly preferred is polyvinylpyrrolidone of a molecular weight of about 50,000. In the pharmaceutical composition according to the invention polyvinylpyrrolidone (PVP) is also a binding agent.

The content of the hydrophilic component in the tablet's core depends on the type of the substance used. For polyvinylpirrolidones, their content depends on their molecular weight and solubility associated therewith as well as on other physical properties. It could be selected by those skilled in the art on the basis of PVP characteristics and a common knowledge.

In case of using polyvinylpyrrolidone of a molecular weight of about 50,000, its fraction is preferably 5-12% by weight based on the weight of the tablet's core.

The assumed profile of controlled-release of tamsulosin hydrochloride is achieved at the weight ratio of the fatty and hydrophilic components of the tablet's core within the range from 2:1 to 6:1, preferably at about 4:1.

The core of the pharmaceutical composition according to the invention further comprises at least one diluent that can be any substance increasing the bulk of the tablet.

In a preferred embodiment, two different diluents of complementary properties are used, whereby one of them facilitates penetration of water into the tablet's core while the other provides its skeleton. Appropriate diluents are selected from the group consisting of sugar alcohols, such as mannitol, sorbitol, xylitol or maltitol; sugars, such as glucose, lactose, levulose, sucrose, maltose, glucose and dextrose; starches, such as corn starch and potato starch; cellulose derivatives, such as microcrystalline cellulose; cellulose acetate; colloidal silica; calcium sulfate; di- and tri-basic calcium phosphate; calcium carbonate; nonpareils; talc and other.

Preferred diluents in the pharmaceutical composition according to the invention are a combination of sorbitol and lactose.

Sorbitol, hexane-1,2,3,4,5,6-hexyl, is used as a diluent in tablet formulations prepared by either wet granulation or direct compression. It is available in an amorphous form as well as in four crystalline polymorphic forms, and provides the bulk of the tablet as well as facilitates the erosion of the core skeleton by water.

Lactose, (O-β-D-galactopyranosyl-(1→4)-α-D-glucopyranose), that is less soluble in water than sorbitol, is widely used as a filler or diluent in tablets and capsules. It is available in two anomeric forms, α and β, among which β-lactose is anhydrous whereas α-lactose is anhydrous or hydrated, having various distribution of molecular weight and flow characteristics. In the pharmaceutical composition according to the invention, lactose monohydrate is preferred due to its highly porous structure and a large specific area.

The core of the tablet according to the invention can further comprise other pharmaceutically acceptable excipients that facilitate the manufacturing process and provide required physical and mechanical properties of the tablet. The additional excipients could be further components of the matrix, such as cellulose derivatives or acrylic polymers; binders such as pre-gelatinized starch; sodium alginate, polyvinyl alcohol, acacia gum; disintegrants such as methylcellulose, low-substituted hydroxypropylcellulose, sodium carboxymethylcellulose, calcium carboxymethylcellulose, guar gum, crosspovidone, croscarmellose sodium, colloidal silicon dioxide, alginic acid, potassium polyacrylate, sodium starch glycolate; hydrophobic agents, such as waxes; preservatives; colorants and other substances, as needed.

The invention relates also to a process for the preparation of the controlled-release formulation of tamsulosin hydrochloride, which process comprises:

(1) wet-granulating the blend of the fatty and hydrophilic matrix components, diluent(s) and binder with an aqueous suspension of tamsulosin hydrochloride to form granules;

(2) drying the granules to remove water;

(3) sieving the granules to standardize their size;

(4) admixing the granules with the additional excipients;

(5) compressing the granules mixture into the cores of the tablets; and

(6) coating the cores of the tablets with an acid-resistant coating film.

The additional excipients may be added to the composition of a core either before preparing granules comprising the active ingredient or to the granules directly before compressing them into the tablet cores. In particular, lubricant(s), glidant(s), additional fillers and binders, if any, are being admixed with the dry granules directly before compressing them into the tablet cores.

Colloidal silicon dioxide, magnesium trisilicate, starch, powdered cellulose, tribasic calcium phosphate, or talc may be used as glidants, which can also play a role of lubricant. The lubricants can be also calcium stearate, magnesium stearate, zinc stearate, sodium lauryl fumarate, hydrogenated castor oil, hydrogenated vegetable oil, and others.

Many of the excipients may play more than one function in the pharmaceutical composition according to the invention.

In general, the additional fillers used in the compression process can be the same fillers which are used to form granules or can be other substances. Preferably, sorbitol instant is used as the filler with hydrophilic properties.

The content of tamsulosin hydrochloride in the solid oral pharmaceutical composition according to the invention is 0.4 mg or its multiplicity per the unit dosage form.

In the preferred embodiment of the invention, the pharmaceutical composition comprises about 0.2 wt % of tamsulosin hydrochloride, 20-40 wt % of a fatty component and 5-12 wt % of a hydrophilic component of the matrix, based on the weight of the tablet's core, made up to 100% with diluents and other excipients.

In the particularly preferred embodiment of the invention, the core of a tablet comprises, in wt % of the core, 0.2% of tamsulosin hydrochloride, 20-40% of glycerol behenate, 5-12% of PVP, 20-40% of lactose, 30-50% of sorbitol (powder and instant forms combined) and 0.5-1.5% of magnesium stearate, made up to 100% with diluents and other excipients.

The core of the tablet is protected by a coating which is resistant to gastric fluids and dissolves only in a neutral to weakly-alkaline medium of the intestine. The coatings of this type are described, for example, in Pharmaceutical Dosage Forms and Drug Delivery Systems, H. C. Ansel, I. V. Allen, N. G. Popovich, 7th ed. (1999), Lippincot, Williams & Wilkins. The acid resistant coatings are formed by anionic polymers and copolymers of acrylic acid or methacrylic acid, with (meth)acrylic acid esters, in particular copolymers of methacrylic acid with methyl methacrylate having free acid units; phthalates, such as, e.g., cellulose acetate phthalate, cellulose polyacetate phthalate, acetylvinyl polyphthalate, acetylcellulose succinate, copolymers of vinyl acetate and crotonic acid, together with additives such as plasticizers, fillers, dispersing agents, colorants and polishing agents.

The appropriate dissolution profile of tamsulosin hydrochloride in the targeted place of gastrointestinal tract is achieved by covering the cores with copolymers of methacrylic acid and esters thereof, such as those known under the trade name Eudragit, for example, Eudragit S100 or Eudragit L 30 D-55 or mixtures thereof. Eudragit L 30 D-55 is a water dispersion of a copolymer based on methacrylic acid and acrylic acid ethyl ester having the ratio of free carboxyl groups to the ester groups of approx. 1:1. Eudragit S 100 is a copolymer of methacrylic acid and methacrylic acid methyl ester having the ratio of free carboxyl groups to the ester groups of approx. 1:2 and it needs to be partially neutralized with aqueous ammonia before coating the cores.

Typically, the coating comprises 2-12% by weight of the tablet core.

The solid oral controlled-release pharmaceutical composition of tamsulosin hydrochloride in the form of enteric-coated tablet according to the invention is characterized by appropriate physicochemical parameters and by an adequate release profile of the active ingredient in vitro.

The dissolution profile of tamsulosin hydrochloride from the pharmaceutical composition has been measured in a Type II paddle apparatus in accordance with the dissolution testing method specified in the European Pharmacopoeia, i.e., at 37±0.5° C. and 100 rpm in the following two steps:
I. In a 0.1 M solution of hydrochloric acid with 0.003% of Tween—after 2 h the dissolution of the active ingredient should be 10-40% of the declared amount;
II. In a phosphate buffer pH 7.2—the dissolution of the active ingredient should be
   35-70% dissolution after 3 h; and
   not less than 70% dissolution of the declared amount after 5 h.

The dissolution test is performed using 6 tablets placed in 6 separate vessels. Each tablet is placed in a vessel of the apparatus containing 500 mL of hydrochloric acid (0.1 mol/L), pre-heated to 37±0.5° C. After covering the vessels with lids, the agitator is turned on. After 2 hours of stirring, 20 mL of the solution is taken out and the amount of dissolved tamsulosin hydrochloride is determined by HPLC using a UV detector at the wavelength λ=225 nm. Next, the whole volume of the acid is poured out from the vessels and is replaced with 500 mL of a pH 7.2 phosphate buffer, pre-heated to 37±0.5° C. In pre-defined time intervals (i.e., after 3 and 5 hours) 10 mL samples are taken out, each time adjusting volume in each vessel to 500 mL with a phosphate buffer having the same pH and temperature. For each sample, the content of dissolved tamsulosin hydrochloride is determined by HPLC using a UV detector at the wavelength λ=225 nm.

The dissolution profiles of the tablet formulation according to the invention and the reference product Omnic® capsules was compared for similarity by calculating a similarity factor. In accordance with guidelines of Committee for Proprietary Medicinal Products (Note for Guidance on Quality of Modified Release Products), the similarity factor is calculated by the following formula:

$${f}_{2} = 50 \times log \{ {[ 1 + ( \frac{1}{n} ) {\sum }_{t = 1}^{n} {( {R}_{t} - {T}_{t} )}^{2} ]}^{- 0.5} \times 100 \}$$

where, f₂ is the similarity factor; n is the number of time points; Rₜ is the mean percent active ingredient dissolved of the reference product; and Tₜ is the mean percent active ingredient dissolved of the pharmaceutical composition according to the invention.

The similarity factor f₂ for the tablets according to the invention was found to be about 70. Accordingly, per the EMEA guidelines, the dissolution profiles of the tablet formulation according to the invention and of the reference product Omnic® capsules are similar.

The solid preparation according to the invention provides appropriate in vitro dissolution profile of tamsulosin hydrochloride.

### EXAMPLES

The invention is further illustrated hereinbelow by the following non-limiting examples.

### Example 1

### Tablet of Tamsulosin Hydrochloride a 0.4 mg

Composition of the tablet's core (in g/1000 tablets):

| | |
|---|---|
| Tamsulosin hydrochloride | 4.0 |
| Lactose 200/25 | 500.0 |
| Compritol ATO 888 | 560.0 |
| PVP K30 | 140.0 |
| Sorbitol - powder | 500.0 |
| Sorbitol instant | 276.0 |
| Magnesium stearate | 20.0 |
| Total: | 2000.0 |

The excipients were sieved, if necessary, through a 0.5 mm sieve. Lactose, powdered sorbitol, and Compritol ATO 888 were stirred for 4 min. at the speed of a planetary-motion paddle of 300 rpm, until a uniform powders blend was obtained. A suspension for granulation was prepared by dispersing tamsulosin hydrochloride (1 wt % excess with respect to the calculated amount), after sieving it through a 0.3 mm sieve, in water (40 mL per 10,000 tablets). After emptying, the reactor was washed with 50 mL of water that was added to the suspension. The suspension was added to the mixture in the granulator. Granulation has taken 16 min. at 300 rpm of the planetary-motion paddle and 1,500 rpm of the high-speed propeller. After 8 min. of stirring, the speed of the propeller was increased to 3,000 rpm. The granules, obtained in this way were dried in a fluidized-bed dryer at 30° C. for 8 min., to reach moisture content 0.5-2.0 wt %, and then particle size of the granules was standardized using an oscillating granulator provided with a 0.8 mm sieve. After standardization, the granules were weighed. Necessary amounts of magnesium stearate and sorbitol instant were calculated from the weight of the granules. Weighed amounts of both substances, after careful blending in a barrel-shape blender (15 min., 15 rpm), were added to the granules and the mixture was carefully blended and then compressed on a rotary tableting machine, using 8 mm biconvex punches and controlling the weight of the tablets.

After determining their weight, hardness, friability and dissolution rate, the tablet cores, obtained as above were de-dusted and coated in a pan coater, pre-heated to approx. 40° C. The coating suspension comprising (in mg/500 mg):

| | |
|---|---|
| Eudragit S 100 | 60.0 |
| Triacetin | 30.0 |
| Talc | 20.0 |
| Lactose | 6.0 |
| Aqueous ammonia | q.s., |

was prepared by dispersing the calculated amounts of methacrylic acid polymer and triethyl citrate (triacetin) with aqueous ammonia with high-speed stirring, to partially neutralize the —COOH groups, and after that the homogenous suspension of lactose and talc in the remaining volume of water was added.

The coating process was carried out in a pan coater at a temperature of the tablet bed of 40° C., until a uniform coating of 10-12 mg per tablet, was obtained. After completion of coating, the tablets were tentatively dried for about 30 min., by slowing down revolutions of the coater's drum and lowering air temperature at the inlet to 40-50° C. The tablets, spread loosely on trays, were then dried in a tray drier for 2 h at 40° C.

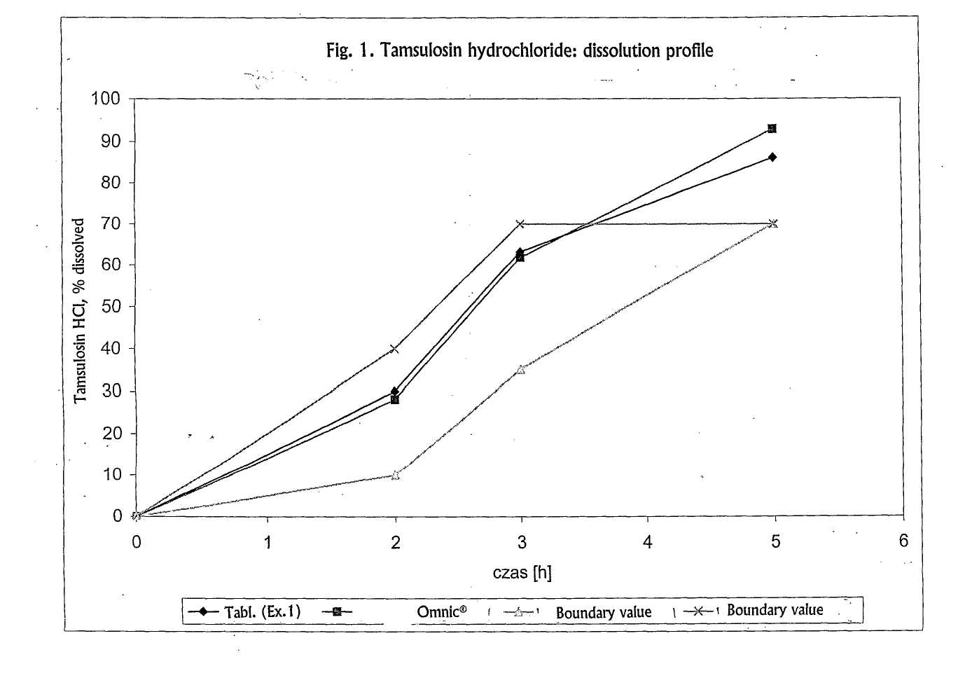

Results of the dissolution test for the pharmaceutical composition of Example 1 and for the reference pharmaceutical composition Omnic® capsules, each comprising 0.4 mg of the active ingredient, are presented in the Table 1 below and in FIG. 1 as a diagram representing the amount of dissolved tamsulosin hydrochloride (in wt %) versus time.

TABLE 1

| | Limits of the | % of the active | |
|---|---|---|---|
| | dissolved | ingredient released | % of the active ingredient |
| Time | active | from Omnic ® | released from the tablets |
| [h] | ingredient [%] | capsules | according to the invention |
| 2 | 10-40 | 28.1 | 30.0 |
| 3 | 35-70 | 61.8 | 63.2 |
| 5 | not less than 70 | 92.9 | 86.2 |

The determination of the dissolution rates of tamsulosin hydrochloride from the tablets prepared as given hereinabove yielded a similarity factor f₂=68.7 with respect to the reference Omnic® capsules.

Stability tests of the tablets prepared as above were conducted over a period of 3 months at 25° C., 60% RH, while accelerated ageing was conducted over a period of 3 months at 40° C., 75% RH. The samples were characterized in terms of physicochemical properties and stability. The results of the tests are presented in Table 2.

TABLE 2

| | Directly after | 3 months | |
|---|---|---|---|
| Parameter | preparation | 25° C., 60% RH | 40° C., 75% RH |
| Appearance | White round | No changes | No changes |
| | tablets, | | |
| | biconvex | | |
| Average weight | 200.0 mg | 202.1 mg | 200.7 mg |
| Purity (by HPLC) | <0.1 | <0.1 | <0.1 |
| Any single | | | |
| impurity ≦0.1 | | | |
| Sum of | <0.5% | <0.5% | <0.5% |
| impurities <0.5% | | | |
| Assay of | 0.39 mg | 0.40 mg | 0.41 mg |
| tamsulosin | | | |
| hydrochloride | | | |
| Dissolution rate: | | | |
| after 2 h | 27.7% | 23.0% | 20.9% |
| after 3 h | 47.5% | 45.7% | 49.6% |
| after 5 h | 84.9% | 85.1% | 80.0% |

### Example 2

Cores of the tablets obtained as in Example 1 were coated in an analogous manner using a 30 wt % aqueous coating dispersion, containing (in g per 10,000 tablets):

| | | |
|---|---|---|
| Eudragit L30 D-55 | 113.3 | g (34.0 g of a solid) |
| Triethyl citrate | 3.4 | g |
| Talc | 5.0 | g |
| Titanium dioxide | 2.5 | g |
| Yellow lake | 0.39 | g. |

The cores have been placed in the drum of the pan coater and de-dusted. The bed of the cores has been heated up to about 30-34° C. with inlet air temperature 45° C. and then the cores were coated by a uniform stream of the dispersion. Average weight increase of the coating has been controlled, until it reached approximately 4.6 mg per tablet. After coating, the tablets were dried for 5 min. at 35° C. (temperature of inlet air).

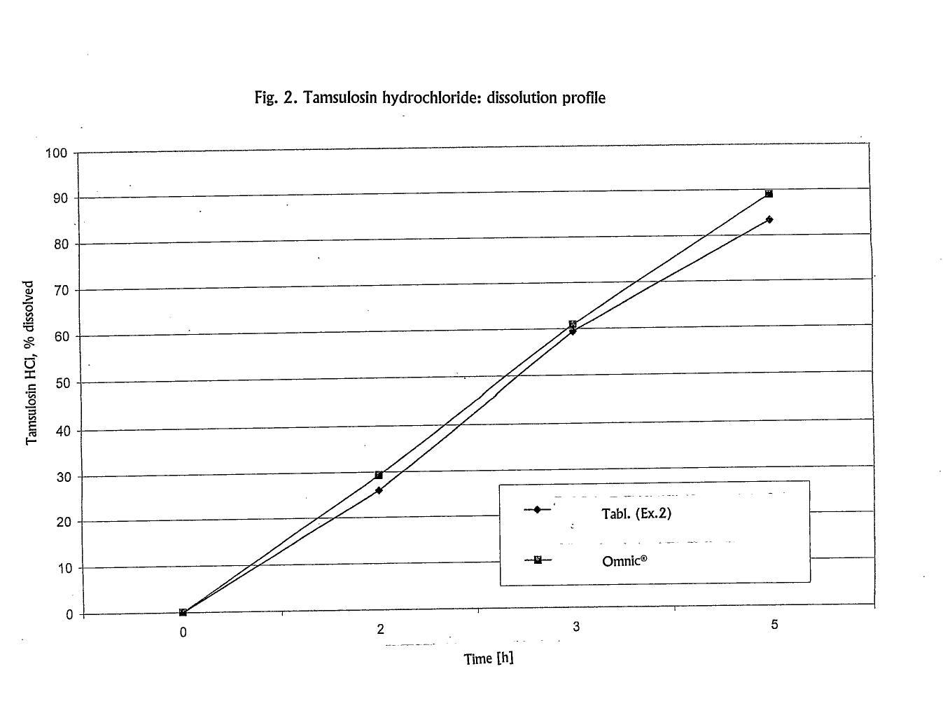

Determination of the dissolution rates of tamsulosin hydrochloride from the tablets prepared as hereinabove yielded a similarity coefficient f₂=70.01 with respect to the reference Omnic® capsules. The dissolution profiles of both pharmaceutical compositions are presented in FIG. 2.

1-21. (canceled)

22. A pharmaceutical composition for oral administration comprising tamsulosin hydrochloride and at least one diluent, and optionally one or more other pharmaceutically acceptable excipients, wherein tamsulosin hydrochloride is homogenously dispersed within a matrix comprising a mixture of a fatty component and a hydrophilic component, and the pharmaceutical composition is in the form of an enteric-coated tablet.

23. The pharmaceutical composition according to claim 22, which when subjected to a 0.1 N HCl buffer for 2 hours, followed by a pH 7.2 buffer for 5 hours, at 37±0.5° C., and agitation during the 7 hours by a Type II paddle apparatus at 100 rpm, has released
a total of 10-40% by weight of tamsulosin hydrochloride during the 2 hours in the HCl buffer,
a total of 35-70% of tamsulosin hydrochloride by weight during the 2 hours in the HCl buffer and initial 3 hours in the pH 7.2 buffer, and
a total of not less than 70% of tamsulosin hydrochloride by weight during the 2 hours in the HCl buffer and the 5 hours in the pH 7.2 buffer.

24. The pharmaceutical composition according to claim 23, wherein said fatty component is a fatty acid glyceride.

25. The pharmaceutical composition according to claim 24, wherein said fatty acid glyceride is selected from the group consisting of C12-C18 long-chain fatty acids, glycerides of C₈-C₁₈ medium- and long-chain fatty acids; hydrogenated fatty oils; hydrogenated lecithins; and the mixtures thereof.

26. The pharmaceutical composition according to claim 25, wherein the fatty acid glyceride is glycerol behenate.

27. The pharmaceutical composition according to claim 22, wherein the weight ratio of said fatty component to said hydrophilic component is between 2:1 and 6:1.

28. The pharmaceutical composition according to claim 27, wherein the weight ratio of said fatty component to said hydrophilic component is about 4:1.

29. The pharmaceutical composition according to claim 22, wherein said hydrophilic component is selected from the group consisting of polyvinylpyrrolidone, polyvinyl alcohols, polyethylene glycols, ethers and esters of cellulose, hydroxypropylmethylcellulose, sodium carboxymethylcellulose, alginates, and the mixtures thereof.

30. The pharmaceutical composition according to claim 29, wherein said hydrophilic component is polyvinylpyrrolidone.

31. The pharmaceutical composition according to claim 30, wherein said hydrophilic component is a polyvinylpyrrolidone having a molecular weight within the range of 25,000 and 300,000.

32. The pharmaceutical composition according to claim 31, wherein said hydrophilic component is a polyvinylpyrrolidone having a molecular weight of about 50,000.

33. The pharmaceutical composition according to claim 22, comprising a tablet core and a tablet coating, wherein said core comprises two diluents having complementary properties.

34. The pharmaceutical composition according to claim 33, wherein said diluents are lactose and sorbitol.

35. The pharmaceutical composition according to claim 22, optionally comprising additional matrix components, binders, lubricants, glidants, colorants, and/or other pharmaceutically acceptable excipients.

36. The pharmaceutical composition according to claim 22, comprising tamsulosin hydrochloride in the amount of 0.4 mg per unit dosage form.

37. The pharmaceutical composition according to claim 33, comprising, in wt % of said tablet core, 0.2% of tamsulosin hydrochloride, 20-40% of glycerol behenate, 5-12% of polyvinylpyrrolidone, 20-40% of lactose, 30-50% of sorbitol and 0.5-1.5% of magnesium stearate.

38. The pharmaceutical composition according to claim 33, wherein said tablet coating comprises a copolymer of methacrylic acid.

39. The pharmaceutical composition according to claim 38, wherein said tablet coating consists of a copolymer of methacrylic acid and acrylic acid ethyl ester.

40. The pharmaceutical composition according to claim 33, wherein the weight ratio of said tablet coating to said tablet core is about 2-12%.

41. A method for the preparation of the pharmaceutical composition of claim 22, comprising
(1) wet-granulating a blend of a fatty component and a hydrophilic component, and one or more diluents and binders, with an aqueous suspension of tamsulosin hydrochloride to form granules;
(2) drying said granules to remove water;
(3) sieving said granules to standardize their size;
(4) admixing said granules with one or more additional excipients;
(5) compressing said granules mixture into tablet cores; and
(6) coating said tablet cores with an acid-resistant coating.

42. The method according to claim 41, in which said additional excipient is sorbitol instant.
